SEQUENCE LISTING (12 sequences)

SEQ ID NO: 1 - protein, 165 aa, Homo sapiens
WSNNYAVDCPQHCDSSECKSSPRCKRTVLDDCGCCRVCAAGRGETCYRTVSGMDGMKCGPGLRCQPSNGEDPFGEEFGICKDCPYGTFGMDCRETCNCQSGICDRGTGKCLKFPFFQYSVTKSSNRFVSLTEHDMASGDGNIVREEVVKENAAGSPVMRKWLNPR

SEQ ID NO: 2 - protein, 165 aa, Mus musculus
WSAKYAVDCPEHCDKTECRSSLRCKRTVLDDCGCCQVCAAGPGETCYRTVSGMDGVKCGPGLKCHFYSEEDDFGDEFGICKDCPYGTFGMECKETCNCQSGICDRVTGRCLDFPFFQYAAAKSPSRTSASHTERDSASGDGNAVREEIGEGNAARPSVMKWLNPR

SEQ ID NO: 3 - protein, 184 aa, Homo sapiens
MKSVLLLTTLLVPAHLVAAWSNNYAVDCPQHCDSSECKSSPRCKRTVLDDCGCCRVCAAGRGETCYRTVSGMDGMKCGPGLRCQPSNGEDPFGEEFGICKDCPYGTFGMDCRETCNCQSGICDRGTGKCLKFPFFQYSVTKSSNRFVSLTEHDMASGDGNIVREEVVKENAAGSPVMRKWLNPR

SEQ ID NO: 4 - protein, 184 aa, Mus musculus
MKSLLLLTTLLVPLHLGMAWSAKYAVDCPEHCDKTECRSSLRCKRTVLDDCGCCQVCAAGPGETCYRTVSGMDGVKCGPGLKCHFYSEEDDFGDEFGICKDCPYGTFGMECKETCNCQSGICDRVTGRCLDFPFFQYAAAKSPSRTSASHTERDSASGDGNAVREEIGEGNAARPSVMKWLNPR

SEQ ID NO: 5 - protein, 165 aa, Homo sapiens
WSNNYAVDCPQHCDSSECKSSPRCKRTVLDDCGCCRVCAAGRGETCYRTVSGMDGMKCGPGLRCQPSNGEDPFGEEFGICKDCPYGTFGMDCRETCNCQSGICDRGTGKCLKFPFFQYSVTKSSNRFVSLTEHDMAAGDGNIVREEVVKENAAGSPVMRKWLNPR

SEQ ID NO: 6 - protein, 165 aa, Mus musculus
WSAKYAVDCPEHCDKTECRSSLRCKRTVLDDCGCCQVCAAGPGETCYRTVSGMDGVKCGPGLKCHFYSEEDDFGDEFGICKDCPYGTFGMECKETCNCQSGICDRVTGRCLDFPFFQYAAAKSPSRTSASHTERDSAAGDGNAVREEIGEGNAARPSVMKWLNPR

SEQ ID NO: 7 - protein, 165 aa, Homo sapiens
WSNNYAVDCPQHCDSSECKSSPRCKRTVLDDCGCCRVCAAGRGETCYRTVSGMDGMKCGPGLRCQPSNGEDPFGEEFGICKDCPYGTFGMDCRETCNCQSGICDRGTGKCLKFPFAQYSVTKSSNRFVSLTEHDMAAGDGNIVREEVVKENAAGSPVMRKWLNPR

SEQ ID NO: 8 - protein, 165 aa, Mus musculus
WSAKYAVDCPEHCDKTECRSSLRCKRTVLDDCGCCQVCAAGPGETCYRTVSGMDGVKCGPGLKCHFYSEEDDFGDEFGICKDCPYGTFGMECKETCNCQSGICDRVTGRCLDFPFAQYAAAKSPSRTSASHTERDSAAGDGNAVREEIGEGNAARPSVMKWLNPR

SEQ ID NO: 9 - DNA, 555 bp, Homo sapiens
atgaagagcgtcttgctgctgaccacgctcctcgtgcctgcacacctggtggccgcctggagcaataattatgcggtggactgccctcaacactgtgacagcagtgagtgcaaaagcagcccgcgctgcaagaggacagtgctcgacgactgtggctgctgccgagtgtgcgctgcagggcggggagaaacttgctaccgcacagtctcaggcatggatggcatgaagtgtggcccggggctgaggtgtcagccttctaatggggaggatccttttggtgaagagtttggtatctgcaaagactgtccctacggcaccttcgggatggattgcagagagacctgcaactgccagtcaggcatctgtgacagggggacgggaaaatgcctgaaattccccttcttccaatattcagtaaccaagtcttccaacagatttgtttctctcacggagcatgacatggcatctggagatggcaatattgtgagagaagaagttgtgaaagagaatgctgccgggtctcccgtaatgaggaaatggttaaatccacgctga

SEQ ID NO: 10 - DNA, 555 bp, Mus musculus
atgaagagcctcttgctgctgaccacactcctggtacctctgcacctgggcatggcctggagcgccaaatatgcggtggattgcccggaacattgtgacaaaactgagtgcagaagcagcctgcgttgcaagaggacagtgctggatgactgtggctgctgccaggtgtgcgccgcgggacctggagaaacctgctaccgtacagtctcaggcatggacggggtcaagtgtggtccggggctgaagtgtcacttttacggcgaggaggatgattttggtgacgagtttggtatctgcaaagactgtccctatggcaccttcgggatggaatgcaaagagacttgcaattgccagtcgggcatatgtgacagggtgaccggaagatgtctggactttcccttcttccagtatgcagcagccaaatctcccagcaggacctctgcctcccacacagagcgtgactcagcatccggagatggcaacgctgtgagagaagagattggagaagggaacgctgctcggccctccgtgatgaaatggttaaatccacgctga

SEQ ID NO: 11 - DNA, 15 bp, Artificial sequence
  Synthetic Primer
agaaacttgctaccg

SEQ ID NO: 12 - DNA, 15 bp, Artificial sequence
  Synthetic Primer
gccgtagggacagtc

(19) United States
(12) Patent Application Publication
(10) Pub. No.: US 2011/0301092 A1
(43) Pub. Date: Dec. 8, 2011

(54) THE USE OF A NON-GLYCANATED POLYPEPTIDE FOR TREATING A CANCER

(75) Inventors: Philippe Lassalle, Lille Cedex (FR); Bogdan Grigoriu, Lille Cedex (FR); Florence Depontieu, Lille Cedex (FR)

(73) Assignee: INSTITUT PASTEUR DE LILLE, Lille (FR)

(21) Appl. No.: 12/739,755

(22) PCT Filed: Oct. 24, 2008
(86) PCT No.: PCT/EP08/64449

§ 371 Date: Jun. 7, 2011

(30) Foreign Application Priority Data
Oct. 24, 2007 (EP) 07301488.8

(51) Int. Cl. A61K 38/17; C07H 21/00; A61P 35/00; C12N 5/10; C12N 1/00; C07K 14/47; C12N 15/63

(52) U.S. Cl. 514/19.3; 530/350; 536/23.5; 435/320.1; 435/325; 435/348; 435/358; 435/365; 435/364; 435/352; 435/366; 435/367; 435/370; 435/243

(57) ABSTRACT

The use of a non-glycanated form of a polypeptide including an amino acid sequence having at least 90% amino acid identity with an amino acid sequence selected from the group consisting of SEQ ID No 1 and SEQ ID No 2 for manufacturing a medicament for preventing or treating a cancer.

### FIELD OF THE INVENTION

The present invention relates to the field of the medical treatment of cancers, including the treatment of cancers with polypeptides.

### BACKGROUND OF THE INVENTION

In 2000, worldwide, there were more than 10 million cases of cancer identified, and over 6 million cancer-related deaths. 23% of all deaths in the United States in 2000 were cancer-related.

The increased number of cancer cases reported around the world, is a major concern. Currently there are only a handful of treatments available for specific types of cancer, and these provide no absolute guarantee of success. Among them, ovarian cancer is the fifth most common cancer (other than skin cancer) in women. It ranks fifth as the cause of cancer death in women. The American Cancer Society estimates that there will be about 25,580 new cases of ovarian cancer in this country in 2004. About 16,090 women will die of the disease.

Despite advances in the chemotherapy, surgery and supportive care, death rates for cancer disease have remained constant for nearly two decades (National Cancer Institute. SEER Cancer. Statistics Review 1973-1997, 2001). New diagnostic methods and therapies are thus needed.

Also, because almost all currently available antineoplastic agents have significant toxicity, such as bone marrow suppression, renal dysfunction, stomatitis, enteritis and hair loss, it would be of major advantage to have a relatively less toxic agent available for use alone or in combination with current drugs in order to better treat the patient, preferably without risking injury caused by the therapy itself.

### SUMMARY OF THE INVENTION

This invention relates to the use of a non-glycanated form of a polypeptide comprising an amino acid sequence having at least 90% amino acid identity with an amino acid sequence selected from the group consisting of SEQ ID No 1 and SEQ ID No 2 for manufacturing a medicament for preventing or treating a cancer.

The present invention notably pertains to the use of a polypeptide comprising an amino acid sequence having at least 90% amino acid identity with an amino acid sequence selected from the group consisting of SEQ ID No 1 and SEQ ID No 2, which polypeptide is mutated on one or more amino acid residues involved in its glycosylation, for manufacturing a medicament for preventing or treating a cancer.

This invention also concerns a pharmaceutical composition comprising such a mutated polypeptide that comprises an amino acid sequence selected from the group consisting of SEQ ID No 5, 6, 7 and 8.

The present invention also deals with nucleic acids and expression cassettes encoding the mutated polypeptides defined above, as well as with corresponding recombinant vectors and recombinant host cells, that may also be used themselves as medicinal agents against cancer.

### BRIEF DESCRIPTION OF THE FIGURES

FIG. 1. Transfected HT29 cells in SCID mouse.

SCID mice were injected with 0.2×10⁶ cells of human endocan—(black triangle), mouse endocan—(black square), unglycanable human endocan—(white triangle), unglycanable mouse endocan—(white square), or control vector—(star) transfected HT29 cells (n=4 per group). Four independents clones of endocan-HT29 transfected cell lines were tested. Results present one clone representative of the others clones. Tumor growth was analyzed in each experiment by measurement of tumour size once a week. Mice were euthanized when the tumour volume reached 1000 mm³. Abscissa: time period after cell injection, expressed as weeks. Ordinate: volume of the tumor expressed as mm³. The results are depicted as median±interquartiles.

FIG. 2. Effect of endocan on HT29 cell proliferation in vitro.

BrDU and MTT ODs after 24 hours of HT29 culture with medium, mytomycine (100 μg/mL), or various quantities of endocan ranges from 0.001 to 1 μg/mL. Human and mouse wild type and unglycanable endocan were tested. Ordinate: Absorbance value, expressed as O.D. The results are depicted as median±FIG. 3: Kinetics of mouse endocan-HT-29 cells in SCID mouse.

SCID mice were injected with 0.2×10⁶ cells of endocan—(black box), or control vector-(white box) transfected HT-29 cells (n=4 per group) in two independent experiences. The first one (lozenge) Four independents clones of mouse endocan-HT-29 transfected cell lines were tested. Tumor growth was analyzed in each experiment by measurement of tumor size once a week. Mice were euthanized when the tumor volume reached 1000 mm³. Abscissa: time period after cell injection, expressed as weeks. The results are depicted as median±interquartiles. A: Mouse endocan-HT-29 cells; B: Mouse endocan/S138A-HT-29 cells; C: Human endocan/S137A-HT-29 cells.

FIG. 4. The growth rate of HT29 overexpressing unglycanated endocan is not dependent of cell clone.

Three separate HT29 cell clones overexpressing E1 or E11 were subcutaneously injected into SCID mice (2×10⁵ cells per mouse, 4 mice per clone). Mice were examined each week. Mice were sacrified at week 7 and tumours analysed microscopically. Mean+/−SD of mean of the 3 clones (12 mice).

FIG. 5. Role of F115 and F116 in the anti-tumour activity of unglycanated human endocan.

Three HT29 cell clones (obtained by limited dilution) overexpressing E11, E15, E16 or E17 were subcutaneously injected in SCID mice (4 mice per clone). Mice were examined each. Mice were sacrified at week 7 and tumours analysed microscopically. Mean+/−SD of mean of the 3 clones (12 mice).

FIG. 6. Blood levels of endocan in mice bearing HT29 transfected tumours.

Blood E1 and E11 were measured by standard ELISA specific for human endocan. The results are mean+/−SD.

FIG. 7. Blood levels of endocan in mice bearing HT29 transfected tumours. Blood E11, E15, E16, E17 were measured by ELISA specific for human endocan. The results are mean+/−SD.

FIG. 8. The double tumour model. Growth rate of source tumours.

The double tumour model was developed to study the effect of systemic administration of E16 on HT29 tumours. This model consists in simultaneous injection of HT29 cells overexpressing E16 into right scapular region (Source tumours), and injection of parental HT29 cells in the controlateral back (Target tumours) (n=4, mean+/−SD). The controls were made of parental HT29 (n=4), HT29 overexpressing E1 (n=4) or E11 (n=4) as source tumours. Mice were examined each week. Mice were sacrified at week 7 and tumours analysed microscopically. Mean+/−SD. Abscissa: time period after cell injection, expresses as days. Ordinate: tumor volume, expresses as mm³.

FIG. 9. The double tumour model. Growth rate of target tumours.

Mice were examined each week. Mice were sacrified at week 7 and tumours analysed microscopically. Mean+/−SD. Abscissa: time period after cell injection, expresses as days. Ordinate: tumor volume, expresses as mm³.

### DETAILED DESCRIPTION OF THE INVENTION

Surprisingly, it has been found according to the invention that a non-glycanated form of a polypeptide known in the art as “Endocan” or “ESM1” possesses anti-cancer or anti-tumor properties.

More precisely, it has been shown according to the invention that a non-glycanated form of Endocan has the ability to inhibit the growth of tumors in vivo.

Human Endocan was previously known as an endothelial cell-derived glycoprotein that was found at high concentration in the plasma of patients affected with a cancer, particularly of patients affected with a lung, kidney, breast or a vascular endothelial cancer.

It had been shown in the art that the intact glycosylated human Endocan increased the in vitro mitogenic activity of HGF/SF (“Hepatocyte Growth Factor/Scatter Factor”) towards human kidney non-tumor cells, whereas the unglycanated form of Endocan had no effect. Although the in vivo effect of Endocan was not known, a role of this protein in the regulation of HGF/SF, as well as in areas of embryonic development, tissue regeneration, or tumor progression was hypothesized (See Bechard et al., 2001, J Biol Chem, Vol. 278(N^{o} 51): 48341-48349). It had further been shown in the art that Endocan was pro-tumorigenic, since the non-tumorous human kidney cell line HEK293 was induced to form tumors in vivo in SCID mice, after having been transfected by the human endocan cDNA. It had also been shown that both the glycan and a phenylalanine-rich region of Endocan were necessary for exerting Endocan's pro-tumorigenic activity. Notably, it was shown that the expression by transfected HEK293 cells of glycosylated mutated Endocans ((i) F116A and (ii) F115A-F116A, respectively) did not induce in vivo tumor formation despite the presence of the glycan. Thus, Endocan's F115 and F116 residues were shown to mediate tumor growth of initially non-tumorigenic cells. From these results, it was hypothesized that Endocan might represent in the future an original and novel target for anticancer therapy, as well as a marker for some kinds of solid tumors (See Scherpereel et al., 2003, Cancer Research, Vol. 63: 6084-6089).

Also, The PCT application published under n^{o} WO 02/38178 disclosed an Endocan-specific monoclonal antibody, named “MEP-08”, which increased the survival time of mice in which tumors were experimentally induced with HEK 293 cells recombinantly expressing Endocan. The PCT application n^{o} WO 02/38178 also disclosed mutated glycosylated Endocans polypeptides and peptides wherein one or both of the F115 and F116 amino acid residues were replaced by an alanine residue. These mutated glycosylated endocans were described as potential ESM-1 (i.e. Endocan) antagonist compounds.

Thus, human Endocan was known in the art as a pro-tumorigenic protein for initially non-tumor cells. It was also known that both the glycan moiety and a phenylalanine-rich region were involved in the Endocan's tumorigenic activity, which thus might be used as a target protein for designing novel cancer treatments. Also, mutated forms of the glycosylated human Endocan were suggested for use as antagonists of natural Endocan.

Prior art in vitro analysis of endocan on HEK293 cell proliferation indicated that:
endocan alone had no effect but increased the mitogenic activity of HGF/SF;
the endocan's comitogenic activity is mimicked only by its glycan;
the unglycanated endocan had no effect on cell proliferation either alone or in the presence of HGF/SF.

Further investigations indicated that unglycanated endocan has no effect on HT29 cell proliferation in the presence of foetal calf serum.

Moreover, stable transfection of wild type or unglycanated endocan in HEK293 or

HT29 cells did not modify the growth rate of these cells. Thus from in vitro studies, unglycanated endocan did not modify the growth rate of tumor epithelial cell lines like HEK293 or HT29 cells, and thus unglycanated endocan was suspected to have no anti-tumor activity in xenograft models, as initially described with HEK293 overexpressing unglycanated endocan which did not induce tumors when injected in the skin of SCID mice (Scherpereel et al Cancer Res, 2003).

However, as it has been already mentioned above, it has been surprisingly found according to the invention that an unglycanated form of Endocan is able to inhibit the growth of tumors in vivo.

Notably, it has been shown in the examples herein that cancerous cells do not develop in vivo into tumors if these cells recombinantly express an unglycanated form of Endocan. It has further been shown that a recombinant unglycanated Endocan inhibits the development of target solid tumors both (i) when the said recombinant unglycanated Endocan is present locally and (ii) when the said recombinant unglycanated Endocan is administered via a systemic route. In all cases, a stromal inflammatory reaction was induced at the tumor site in the animals treated with an unglycanated Endocan, which supports the usefulness of an unglycanated Endocan notably as an adjuvant compound to cancer immunotherapy.

The in vivo anti-tumor activity of an unglycanated Endocan has been shown in the examples herein both with an unglycanated form of human Endocan and with an unglycanated form of mouse Endocan. There is 75% amino acid identity between the amino acid sequences of human and mouse Endocans, respectively.

The present invention's findings are all the more surprising that it is shown herein that there is no specific link between the non-tumorigenic properties of several modified forms of Endocan and their inhibition properties against tumors. For instance, the (i) F116A and the (ii) F115A and F116A mutated Endocans were shown in the art to behave identically as non-tumorigenic polypeptides, whereas it has been found herein that F116A unglycanated Endocan is a tumor inhibitor while the F115A, F116A unglycanated Endocan does not possess any tumor inhibition activity.

An object of the present invention consists of the use of a non-glycanated form of a polypeptide comprising an amino acid sequence having at least 90% amino acid identity with an amino acid sequence selected from the group consisting of SEQ ID No 1 and SEQ ID No 2 for manufacturing a medicament for preventing or treating a cancer.

As used herein, a “non-glycanated” or an “unglycanated” polypeptide consists of a polypeptide having no saccharide or polysaccharide moiety that is covalently linked to any one of the amino acid residues that are comprised in the amino acid sequence of the said polypeptide.

In some embodiments, the said non-glycanated polypeptide may be produced by subjecting the corresponding glycanated polypeptide to a deglycanation reaction, preferably using one or more appropriate enzymes, using techniques well known from the one skilled in the art. For instance, a non-glycanated form of a polypeptide of interest may be obtained as the final product of deglycanation of the initially glycanated polypeptide by GAG-degrading enzymes, like chondroitinase ABC, chondroitinase B, chondroitinase ACI, chondroitinase C and heparinase II, such as described by Bechard et al. (2001, J Biol Chem, Vol. 276(N^{o}51): 48341-48349).

However, in preferred embodiments, the said non-glycanated form polypeptide consists of a polypeptide wherein one or more amino acids involved in the glycanation of the corresponding non-mutated polypeptide have been replaced by the same number of distinct amino acids.

The amino acid sequence of SEQ ID No 1 consists of the amino acid sequence of the secreted form of human Endocan having 165 amino acids in length.

The amino acid sequence of SEQ ID No 2 consists of the amino acid sequence of the secreted form of mouse Endocan having also 165 amino acids in length.

As used herein; a polypeptide comprising the amino acid sequence of SEQ ID No 1 or SEQ ID No 2 consists of a polypeptide comprising, from the N-terminal to the C-terminal end:
(i) a N-terminal amino acid sequence having from 0 to 250 amino acid residues in length;
(ii) the amino acid sequence of SEQ ID No 1 or SEQ ID No 2, and
(iii) a C-terminal amino acid sequence having from 0 to 250 amino acid residues in length.

Thus, the N-terminal or the C-terminal amino acid sequences may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199; 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 amino acid residues in length.

Preferably, the N-terminal amino acid sequence (i) above has from 0 to 19 amino acid residues in length.

In some preferred embodiments, the N-terminal amino acid sequence (i) above consists of all or part of the N-terminal sequence of the corresponding non-secreted form of the human or mouse Endocan polypeptide, which human or mouse N-terminal sequence consists of the signal peptide having 19 amino acids in length. The amino acid sequence of the non-secreted form of human Endocan polypeptide consists of the amino acid sequence of SEQ ID No 3 herein. The amino acid sequence of the non-secreted form of the mouse Endican polypeptide consists of the amino acid sequence of SEQ ID No 4 herein. Thus, the N-terminal sequence (i) above have thus preferably 0, 1, 2, 3, 4, 5, 6, 7, 8, 9, 10, 11, 12, 13, 14, 15, 16, 17, 18 or 19 amino acids in length. The C-terminal sequence (iii) above may have 0, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length.

To determine the percent of identity of two amino acid sequences, the sequence are aligned for optimal comparison purposes. For example, gaps can be introduced in one or both of a first and a second amino acid sequence for optimal alignment and non-homologous sequences can be disregarded for comparison purposes.

For optimal comparison purposes, the percent of identity of two amino acid sequences can be achieved with CLUSTAL W (version 1.82) with the following parameters: (1) CPU MODE=ClustalW mp; (2) ALIGNMENT= [symbol] full [symbol] ; (3) OUTPUT FORMAT= [symbol] aln w/numbers [symbol] ; (4) OUTPUT ORDER= [symbol] aligned [symbol] ; (5) COLOR ALIGNMENT= [symbol] no [symbol] ; (6) KTUP (word size)= [symbol] default [symbol] ; (7) WINDOW LENGTH= [symbol] default [symbol] ; (8) SCORE TYPE= [symbol] percent [symbol] ; (9) TOPDIAG= [symbol] default [symbol] ; (10) PAIRGAP= [symbol] default [symbol] ; (11) PHYLOGENETIC TREE/TREE TYPE= [symbol] none [symbol] ; (12) MATRIX= [symbol] default [symbol] ; (13) GAP OPEN= [symbol] default [symbol] ; (14) END GAPS= [symbol] default [symbol] ; (15) GAP EXTENSION= [symbol] default [symbol] ; (16) GAP DISTANCES= [symbol] default [symbol] ; (17) TREE TYPE= [symbol] cladogram [symbol] et (18) TREE GRAP DISTANCES= [symbol] hide [symbol] .

As used herein, amino acid sequences having 90% or more than 90% amino acid identity with a reference sequence encompass those having at least 91%, 92%, 93%, 94%, 95%, 96%, 97%, 98%, 99% or 99.5% amino acid identity with the said reference sequence.

The amino acid differences with the reference sequence may consist of deletion, addition or substitution of one or more amino acids. However, the amino acid differences consist preferably of the addition or substitution of one or more amino acid residues, and even most preferably of the substitution of one or more amino acid residues.

As it has been already indicated above, preferred embodiments of unglycanated Endocan polypeptides encompass human or mouse Endocan polypeptides wherein the amino acid residue bearing the glycosylation site has been replaced by a distinct amino acid residue, selected among the 19 remaining conventional amino acid residues. In the human Endocan polypeptide, the amino acid residue bearing the glycosylation site consists of the Serine residue located at position 137 of SEQ ID No 1. In the mouse Endocan polypeptide, the amino acid residue bearing the glycosylation site consists of the Serine residue located at position 138 of SEQ ID No 2. Another object of the invention consists of the use of a polypeptide comprising an amino acid sequence selected from the group consisting of:
a) an amino acid sequence having at least 90% amino acid identity with the amino acid sequence of SEQ ID No 1 and wherein the serine amino acid residue in position 137 of SEQ ID No 1 is replaced by a distinct amino acid residue; and
b) an amino acid sequence having at least 90% amino acid identity with the amino acid sequence of SEQ ID No 2 and wherein the serine residue in position 138 of SEQ ID No 2 is replaced by a distinct amino acid residue,
   for manufacturing a medicament for preventing or treating a cancer.

In preferred embodiments of the use above, the said amino acid sequences having at least 90% amino acid identity with SEQ ID No 1 or SEQ ID No 2 possess no deletion nor substitution of both of the phenylalanine residues located at the respective positions 115 and 116 in each of SEQ ID No 1 or SEQ ID No 2.

In other preferred embodiments of the use above, the said amino acid sequences having at least 90% amino acid identity with SEQ ID No 1 or SEQ ID No 2 possess no deletion nor substitution of any one of the cysteine residues comprised therein.

According to these other preferred embodiments, an amino acid sequence having at least 90% amino acid identity with SEQ ID No 1 comprises no deletion nor substitution of the cysteine amino acid residues located at positions 9, 18, 32, 35, 46, 58, 64, 80, 83, 92, 96, 98, 103 and 110 of SEQ ID No 1, which cysteine residues are involved in disulfide bridges. Also, according to these other preferred embodiments, an amino acid sequence having at least 90% amino acid identity with SEQ ID No 1 comprises no deletion nor substitution of the cysteine amino acid residues located at positions 9, 13, 18, 24, 32, 34, 35, 38, 46, 58, 64, 80, 83, 92, 96, 98, 103 and 110 of SEQ ID No 1.

According to these other preferred embodiments, an amino acid sequence having at least 90% amino acid identity with SEQ ID No 2 comprises no deletion nor substitution of the cysteine amino acid residues located at positions 9, 18, 32, 35, 46, 58, 64, 80, 83, 92, 96, 98, 103 and 110 of SEQ ID No 2, which cysteine residues are involved in disulfide bridges. Also, according to these other preferred embodiments, an amino acid sequence having at least 90% amino acid identity with SEQ ID No 2 comprises no deletion nor substitution of the cysteine amino acid residues located at positions 9, 13, 18, 24, 32, 34, 35, 38, 46, 58, 64, 80, 83, 92, 96, 98, 103 and 110 of SEQ ID No 2.

It has been shown by the inventors that the in vivo stability of the unglycanated forms of human and mouse Endocans in the blood circulation was similar to that of the corresponding glycanated forms, which findings fully support the usefulness of the polypeptides defined above as sufficiently stable and bioavailable active agents for preventing or treating a cancer. These properties may be due to the conformational stability of the said polypeptides, which comprise several disulfide bridges, as compared for example to peptides having an amino acid length of less than 100 amino acids and even worse less than 50 amino acids. Indicatively, the human unglycanated Endocan of SEQ ID No 1 has a half-lifetime of about one hour when it is administered intravenously as a buffer solution comprising no stabilizing agent. Further, it has been shown according to the invention that superfusion of human unglycanated endocan in SCID mice for one week resulted in detectable and stable levels of blood endocan maintained during all the period of time of superfusion.

Preferably, in a therapeutic polypeptide according to the invention, the Serine residue located at position 137 of SEQ ID No 1 or SEQ ID No 2 is replaced by a distinct amino acid residue consisting of non-aromatic amino acid residues. Non-aromatic residues encompass alanine, leucine, isoleucine, valine, proline, methionine, glycine, serine, threonine, cysteine, asparagine, glutamine, arginine, lysine histidine; aspartic acid and glutamic acid. In certain preferred embodiments of a therapeutic polypeptide according to the invention, the Serine residue located at position 137 of SEQ ID No 1 or SEQ ID No 2 is replaced an alanine residue.

As shown in the examples herein, the anti-tumor activity of an unglycanated Endocan is further enhanced when the phenylalanine residue located at position 116 of SEQ ID No 1 or SEQ ID No 2 is replaced by a distinct amino acid.

Another object of the present invention consists of the use of a polypeptide comprising an amino acid sequence selected from the group consisting of:
a) an amino acid sequence having at least 90% amino acid identity with the amino acid sequence of SEQ ID No 1 and wherein (i) the serine amino acid residue in position 137 of SEQ ID No 1 is replaced by a distinct amino acid residue and (ii) the phenylalanine residue in position 116 of SEQ ID No 1 is replaced by a distinct amino acid residue; and
b) an amino acid sequence having at least 90% amino acid identity with the amino acid sequence of SEQ ID No 2 and wherein the serine residue in position 138 of SEQ ID No 2 is replaced by a distinct amino acid residue and (ii) the phenylalanine residue in position 116 of SEQ ID No 2 is replaced by a distinct amino acid residue, for the manufacture of a medicament for preventing or treating a cancer.

Preferably, in a therapeutic polypeptide according to the invention, the phenylalanine residue located at position 116 of SEQ ID No 1 or SEQ ID No 2 is replaced by a distinct amino acid residue consisting of non-aromatic amino acid residues. In certain preferred embodiments of a therapeutic polypeptide according to the invention, the Serine residue located at position 116 of SEQ ID No 1 or SEQ ID No 2 is replaced an alanine residue.

In certain other embodiments, it is made use of a polypeptide comprising an amino acid sequence selected from the group consisting of:
a) an amino acid sequence having at least 90% amino acid identity with the amino acid sequence of SEQ ID No 1 and wherein (i) the serine amino acid residue in position 137 of SEQ ID No 1 is replaced by a distinct amino acid residue and (ii) the phenylalanine residue in position 115 of SEQ ID No 1 is replaced by a distinct amino acid residue; and
b) an amino acid sequence having at least 90% amino acid identity with the amino acid sequence of SEQ ID No 2 and wherein the serine residue in position 138 of SEQ ID No 2 is replaced by a distinct amino acid residue and (ii) the phenylalanine residue in position 115 of SEQ ID No 2 is replaced by a distinct amino acid residue,

Preferably, in a therapeutic polypeptide according to the invention, the phenylalanine residue located at position 115 of SEQ ID No 1 or SEQ ID No 2 is replaced by a distinct amino acid residue consisting of non-aromatic amino acid residues. In certain preferred embodiments of a therapeutic polypeptide according to the invention, the Serine residue located at position 115 of SEQ ID No 1 or SEQ ID No 2 is replaced an alanine residue.

In certain preferred embodiments, the said anti-tumor polypeptide comprises the amino acid sequence selected from the group consisting of SEQ ID No 5 and SEQ ID No 6. In some other preferred embodiments, the said anti-tumor polypeptide comprises the amino acid sequence selected from the group consisting of SEQ ID No 5 and SEQ ID No 6.

In certain preferred embodiments, the said anti-tumor polypeptide comprises the amino acid sequence selected from the group consisting of SEQ ID No 7 and SEQ ID No 8. In some other preferred embodiments, the said anti-tumor polypeptide comprises the amino acid sequence selected from the group consisting of SEQ ID No 7 and SEQ ID No 8.

The present invention also pertains to an unglycanated polypeptide comprising an amino acid sequence selected from the group consisting of SEQ ID No 7 and SEQ ID No 8. It also concerns an unglycanated polypeptide consisting of an amino acid sequence selected from the group consisting of SEQ ID No 7 and SEQ ID No 8.

Another object of the invention consists of a pharmaceutical composition comprising a polypeptide comprising an amino acid sequence selected from the group consisting of SEQ ID No 5, 6, 7 and 8.

A further object of the invention consists of a pharmaceutical composition comprising a polypeptide consisting of an amino acid sequence selected from the group consisting of SEQ ID No 5, 6, 7 and 8.

In the pharmaceutical compositions defined above, the said polypeptide consists of an active ingredient.

According to a first embodiment, a pharmaceutical composition according to the invention contains a therapeutically effective quantity of an unglycanated Endocan-derived anti-tumor polypeptide as described herein, in combination with one or more pharmaceutically compatible vehicles. The pharmaceutical compositions according to the invention include those suitable for topical, oral, rectal, nasal or parenteral (including intramuscular, subcutaneous and intravenous) administration or in a form suitable for administration by inhalation or insufflation. The pharmaceutical compositions according to the invention may be presented in the form of unit doses and may be prepared by any method well known to a person skilled in the art of pharmaceutical medicine. All the methods include a step consisting of combining the antagonist compound comprising the active principle of the composition with a liquid vehicle or a finely divided solid vehicle and, if necessary, forming the product, for example in the form of tablets or capsules.

For oral administration, a pharmaceutical composition according to the invention is preferably presented in the form of dose units such as tablets, capsules or hard capsules. When it is presented in a form contained in a pressurized container, the pharmaceutical composition may contain a propellant such as dichlorodifluoromethane, trichlorofluoromethane, dichlorotetrafluoroethane, carbon dioxide or other appropriate gases. In the case of a pressurized aerosol, the dose unit may be provided with a valve able to supply a given quantity of the pharmaceutical composition.

According to another embodiment, the pharmaceutical composition according to the invention may be in the form of a dry powder composition for administration by inhalation or insufflation, for example in the form of a mixture of a powder of the antagonist compound and of a suitable base powder, such as lactose or starch. The powder composition may be presented in a dose unit, for example in the form of capsules or dispensers from which the powder may be administered using an inhaler or insufflator device.

A solid pharmaceutically acceptable vehicle compatible with a pharmaceutical composition according to the invention includes substances such as flavouring agents, lubricants, solubilizing agents, suspension agents, fillers, compression auxiliaries, binders or dispersion agents as well as encapsulating materials. In the powders, the vehicle is a finely divided solid which is in admixture with the anti-tumor polypeptide described herein, which is also in a finely divided form. In the tablets, the said active ingredient is mixed with a vehicle having suitable compression properties and compacted into the desired form and size. The powders and tablets preferably contain less than 99% of the active ingredient. The preferred solid vehicles are for example calcium phosphate, magnesium stearate, talc, sugars, lactose, dextrin, starch, gelatine, cellulose, polyvinylpyrrolidone and the ion-exchange resins.

Liquid vehicles are used to prepare a pharmaceutical composition according to the invention in the form of a solution, a suspension, an emulsion, a syrup, an elixir and a pressurized composition. The active ingredient may be dissolved or suspended in a pharmaceutically acceptable vehicle such as water, an organic solvent, or a mixture of the two or pharmaceutically acceptable oils or fats. The liquid vehicle may contain other pharmaceutically acceptable additives such as solubilizing agents, emulsifiers, buffers, preservatives, sweeteners, flavouring agents, suspension agents, thickening agents, colorants, viscosity regulators, stabilizers or osmo-regulators. Illustrative examples of liquid vehicles for oral and parenteral administration include water, alcohols, (including monohydric and polyhydric alcohols such as the glycols), oils such as coconut oil or fractionated peanut oil. For parenteral administration, the vehicle may also be an ester such as ethyl oleate and isopropyl myristate.

Liquid pharmaceutical compositions in the form of sterile solutions or suspensions may be used for intramuscular, intraperitoneal or subcutaneous injection.

In another embodiment of the invention, there are provided pharmaceutical compositions comprising at least one of the polypeptide active ingredients of the invention, in a pharmaceutically acceptable vehicle, for the treatment of cancers.

The pharmaceutical compositions according to the present invention can be used for therapeutic treatment of cancers of any kind or type, including carcinomas, sarcomas and leukaemia

In preferred embodiments, the pharmaceutical compositions of the present invention can be used for therapeutic treatment for cancers selected from the group consisting of lung cancer, breast cancer, kidney cancer, pancreas cancer, colorectal cancer and malignant melanoma.

The pharmaceutical compositions according to the invention may be used in combination with other treatment modalities, such as chemotherapy, cryotherapy, hyperthernia, radiation therapy, and the like.

It is shown herein that any one of the anti-tumor polypeptide active ingredient of the invention inhibits the tumor growth in vivo. In some embodiments, the anti-tumor activity that is exerted by a polypeptide active ingredient of the invention may be completed by an additional anti-cancer treatment.

In other embodiments of the invention, there are provided pharmaceutical compositions comprising at least one the polypeptide active ingredients of the invention in combination with one or more other chemotherapeutic agents, in a pharmaceutically acceptable vehicle, for the treatment of cancers. Examples of chemotherapeutic agents contemplated for use in the practice of this particular invention include Busulfan, Carboplatin, Cisplatin, Cyclophosphamide, Cytosine arabinoside, Etoposide, 5-Fluorouracil, Melphalan, Methotrexate, Mitoxantrone, Taxol, Interferon, Fareston, Arzoxifene, Evista, Tamoxifen, and the like.

A pharmaceutical composition according to the invention preferably contains from 0.001 to 1000 mg of the said polypeptide active ingredient per dose unit, and preferably from 0.1 to 50 mg of antagonist compound of the said polypeptide active ingredient per dose unit.

Generally, a pharmaceutical composition according to the invention comprises from 0.01% to 99.9% by weight of a polypeptide active ingredient defined herein in combination with from 99.99% to 0.01% of one or more pharmaceutically compatible excipient. In most cases, a pharmaceutical composition according to the invention comprises from 1% to 99% by weight of a polypeptide active ingredient defined herein in combination with from 99% to 1% of one or more pharmaceutically compatible excipient.

The present invention also concerns a method of treatment and/or prevention of a cancer comprising a step of administering, to the patient in need thereof, a polypeptide active ingredient such as disclosed in the present specification.

Thus, the present invention also provides a method for the treatment or prevention of a human or animal organism, comprising administering to said organism a therapeutically effective amount of a polypeptide active ingredient described herein. If desired, the method of the invention can be carried out in conjunction with one or more conventional therapeutic modalities (e.g. radiation, chemotherapy and/or surgery). The use of multiple therapeutic approaches provides the patient affected with a cancer with a broader based intervention.

The polypeptide active ingredients of the invention can be prepared by the standard peptide syntheses well known to a person skilled in the art.

The polypeptide active ingredients of the invention can be obtained by the genetic engineering technique which comprises the stages of: (i) culture of a microorganism or of eukaryotic cells transformed using a nucleotide sequence according to the invention and (ii) recovery of the peptide produced by said microorganism or said eukaryotic cells.

This technique is well known to a person skilled in the art. For more details concerning this, reference can be made to the following work: Recombinant DNA Technology I, Editors Ales Prokop, Raskesh K Bajpai; Annals of the New York Academy of Sciences, Volume 646, 1991.

A nucleic acid sequence encoding human Endocan consists of SEQ ID No 9.

A nucleic acid encoding mouse Endocan consists of SEQ ID No 10.

Indeed, the one skilled in the art may easily synthesize or produce any one of the nucleic acid sequences that encode the various unglycanated Endocan-derived polypeptides that are described above in the present specification, using well known recombinant DNA techniques, including site-directed mutagenesis techniques.

Notably, the nucleic acids encoding any one of the polypeptide active ingredients of the invention can be prepared by chemical synthesis and genetic engineering using the techniques well known to a person skilled in the art and described for example in Sambrook J. et al., Molecular Cloning: A Laboratory Manual, 1989.

Another object of the present invention consists of a nucleic acid encoding an anti-tumor polypeptide of the invention selected from the group consisting of:
a) a polypeptide comprising an amino acid sequence selected from the group consisting of SEQ ID No 7 and SEQ ID No 8,
b) a polypeptide comprising an amino acid sequence selected from the group consisting of SEQ ID No 7 and SEQ ID No 8.

A further object of the present invention consists of a nucleic acid encoding an anti-tumor polypeptide of the invention selected from the group consisting of:
a) a polypeptide consisting of an amino acid sequence selected from the group consisting of SEQ ID No 7 and SEQ ID No 8,
b) a polypeptide consisting of an amino acid sequence selected from the group consisting of SEQ ID No 7 and SEQ ID No 8.

The nucleic acids of the invention can be inserted into expression vectors in order to obtain the compositions or the polypeptide active ingredients of the invention.

Thus, another object of the invention consists of the recombinant expression vectors comprising a nucleic acid encoding a polypeptide active ingredient of the invention, as well as the means necessary for its expression. Such means necessary for expression are well known in the art and can vary according to the host cell, the expression vector and the level of expression desired.

As expression vectors, there can be mentioned for example the plasmids, the viral vectors of the vaccine virus type, adenovirus, baculovirus, poxvirus, bacterial vectors of salmonella type, BCG. Such vectors and methods of using and making them are well known in the art (see for example <<Nonviral Vectors for gene Therapy>>, 2001, edited by M. Findeis, Humana Press; <<Adenoviral Vectors for Gene Therapy>>, 2002, edited by Curiel and Douglas, Elsevier Science, Academic Press; and <<Vaccinia Virus and poxyirology>>, 2004, edited by S. Isaacs, Humana Press). The term “viral vector” as used herein encompasses vector DNA as well as viral particles generated thereof by conventional technologies.

In certain embodiments, the vector of the invention is an adenoviral vector. It can be derived from a variety of human or animal sources. Any serotype can be employed from the adenovirus serotypes 1 through 51, with a special preference for human adenoviruses 2 (Ad2), 5 (Ad5), 6 (Ad6), 11 (Ad11), 24 (Ad24) and 35 (Ad35). The cited adenoviruses are available from the American Type Culture Collection (ATCC, Rockville, Md.), and have been the subject of numerous publications describing their sequence, organization and methods of producing, allowing the artisan to apply them (see for example U.S. Pat. No. 6,133,028; U.S. Pat. No. 6,110,735; WO02/40665; WO00/50573; EP 1 016 711; Vogels et al., 2003, J. Virol. 77: 8263-8271). Preferably, the adenoviral vector of the invention is replication-defective (see for example WO94/28152; Lusky et al., 1998, J. Virol 72, 2022-2032). Preferred replication-defective adenoviral vectors are E1-defective with an E1 deletion extending from approximately positions 459 to 3328 or from approximately positions 459 to 3510 (by reference to the sequence of the human adenovirus type 5 disclosed in the GeneBank under the accession number M 73260 and in Chroboczek et al., 1992, Virol. 186, 280-285). The cloning capacity can further be improved by deleting additional portion(s) of the adenoviral genome (all or part of the non essential E3 region or of other essential E2, E4 regions). A nucleic acid of the present invention can be inserted in any location of the adenoviral genome. Preferably, it is inserted in replacement of the E1 region. It may be positioned in sense or antisense orientation relative to the natural transcriptional direction of the region in question.

In certain other embodiments, a recombinant vector of the invention is derived from a poxvirus. It may be obtained from any member of the poxyiridae, in particular canarypox, fowlpox and vaccinia virus, the latter being preferred. Suitable vaccinia viruses include without limitation the Copenhagen strain (Goebel et al., 1990, Virol. 179: 247-266 and 517-563; Johnson et al., 1993, Virol. 196: 381-401), the Wyeth strain and the modified Ankara (MVA) strain (Antoine et al., 1998, Virol. 244: 365-396). The general conditions for constructing recombinant poxvirus are well known in the art (see for example EP 206 920; Mayr et al., 1975, Infection 3: 6-14; Sutter and Moss, 1992, Proc. Natl. Acad. Sci. USA 89: 10847-10851; U.S. Pat. No. 6,440,422). A nucleic acid of the present invention is preferably inserted within the poxyiral genome in a non-essential locus. Thymidine kinase gene is particularly appropriate for insertion in Copenhagen vaccinia vectors (Hruby et al., 1983, Proc. Natl. Acad. Sci USA 80: 3411-3415; Weir et al., 1983, J. Virol. 46: 530-537) and deletion II or III for insertion in MVA vector (Meyer et al., 1991, J. Gen. Virol. 72: 1031-1038; Sutter et al., 1994, Vaccine 12: 1032-1040).

In certain further embodiments, the recombinant vector of the invention can be optionally coupled or complexed to conventional drug delivery systems (e.g. lipid or polymer-based liposomes, nanoparticles, etc. such as those described for example in Mahato et al., 1998, Human Gene Ther. 9: 2083-2099 and Allen et al., 2004, Science 303: 18181822).

By “means necessary for the expression” it is meant any means which make it possible to obtain the peptide or fusion peptide of the invention, such as in particular, a promoter, a transcription terminator, a replication origin and preferably a selection marker.

The promoter used in the context of the invention can be of any origin, e.g. viral, cellular or synthetic and be ubiquitous providing constitutive expression or regulable providing for example specific expression in a particular cell type or under specific conditions. It can further be operably linked to an enhancer. Suitable viral promoters include without limitation early promoters obtained from RSV (Rous Sarcoma Virus), SV40 (Simian Virus), and CMV (Cytomegalovirus; Boshart et al., 1985, Cell 41, 521-530), as well as the TK (Thymidine kinase) promoter of HSV-1 virus (Herpes Virus Simplex-1), the major late adenovirus promoter (MLP) and vaccinia promoters (e.g. 7.5K, HSR, TK, p28, p11 and K1L promoters). One may use synthetic promoters such as those described in particular by Chakrabarti et al. (1997, Biotechniques 23: 1094-1097), Hammond et al. (1997, J. Virological Methods 66: 135-138). Suitable cellular promoters include any promoter driving expression of cellular genes with a special interest for liver specific promoters such as those of phosphoglycero kinase (PGK; Adra et al., 1987, Gene 60: 65-74), albumin (Pinkert et al., 1987, Genes Dev. 1: 268-277), phosphoenol pyruvate carboxy kinase (PEPCK) (Eisenberger et al., 1992, Mol. Cell Biol. 12: 1396-1403), cholesterol 7-alpha hydroylase (CYP-7) (Lee et al., 1994, J. Biol. Chem. 269: 14681-14689), alpha-1 antitrypsin (Ciliberto et al., 1985, Cell 41: 531-540), transferrine (Mendelzon et al., 1990, Nucleic Acids Res. 18: 5717-5721); and facteur IX (U.S. Pat. No. 5,814,716) genes.

The vectors of the invention may also comprise one or more additional means in order to improve the transcription rate or level of the nucleotide sequence of the invention in a given host cell, its stability, nuclear RNA transport and/or translation rate or level of the mRNA. Such means are well known by the skilled person and include for example 5′, 3′ non-coding sequences, intervening sequences, splicing sequences, Shine-Dalgarno sequence, Kozak sequence and initiator methionine.

The expression vectors of the invention can comprise either a single nucleotide sequence coding for any one of the peptides of the invention, or at least two nucleotide sequences, it being understood that each nucleotide sequence codes for a peptide of different type.

Another object of the invention consists of a recombinant host cell transformed with a nucleic acid encoding a polypeptide active ingredient of the invention selected from the group consisting of
a) a polypeptide comprising an amino acid sequence selected from the group consisting of SEQ ID No 7 and SEQ ID No 8,
b) a polypeptide comprising an amino acid sequence selected from the group consisting of SEQ ID No 7 and SEQ ID No 8.

A further object of the invention consists of a recombinant host cell transformed with a nucleic acid encoding a polypeptide active ingredient of the invention selected from the group consisting of
a) a polypeptide consisting of an amino acid sequence selected from the group consisting of SEQ ID No 7 and SEQ ID No 8,
b) a polypeptide consisting of an amino acid sequence selected from the group consisting of SEQ ID No 7 and SEQ ID No 8.

The present invention also pertains to a recombinant host cell transformed with a recombinant vector as described herein.

Indeed, any one of the recombinant host cells of the invention express the corresponding polypeptide anti-tumor active ingredient.

In the context of the invention, the term “transformation” or “transformed” has to be understood as meaning “introduction” or “introduced” in a host cell. Any routine method can be used in the art to “transform” a nucleic acid or a recombinant vector in a host cell, e.g. a microorganism or eukaryotic cell. Such methods include, but are not limited to, microinjection (Capechi et al., 1980, Cell 22, 479-488), CaPO.sub.4-mediated transfection (Chen and Okayama, 1987, Mol. Cell Biol. 7, 2745-2752), DEAE-dextran-mediated transfection, electroporation (Chu et al., 1987, Nucleic Acid Res. 15: 1311-1326), lipofection/liposome fusion (Feigner et al., 1987, Proc. Natl. Acad. Sci. USA 84: 7413-7417), particle bombardement (Yang et al., 1990, Proc. Natl. Acad. Sci. USA 87: 9568-9572), gene guns, transduction as well as viral infection. For example, in the context of the invention, a viral vector can be transformed in the host cell by transfection of its genome or by infection of a viral particle. The term “host cell” should be understood broadly without any limitation concerning microorganisms and eukaryotic cells including isolated cells or cells organized in particular structures such as tissues and organs. The host cells may be of a unique type of cells or a group of different types of cells and encompass cultured cell lines, primary cells and proliferative cells.

As examples of eukaryotic cells, there can be mentioned cells originating from animals such as mammals, reptiles, insects and equivalent. The preferred eukaryotic cells are cells originating from the Chinese hamster (CHO cells), monkey (COS and Vero cells), baby hamster kidney (BHK cells), pig kidney (PK 15 cells) and rabbit kidney (RK13 cells, human osteosarcoma cell lines (143 B cells), human HeLa cell lines and human hepatoma cell lines (Hep G2 cell type), as well as insect cell lines (for example of Spodoptera frugiperda).

The host cells can be supplied in cultures in suspension or in vials, in tissue cultures, organ cultures and equivalent. The host cells can also be from transgenic animals. Host cells of the present invention can be cultured in conventional fermentation bioreactors, flasks, and petri plates. Culturing can be carried out at a temperature, pH and oxygen content appropriate for a given host cell. No attempts to describe in detail the various methods known for the production of polypeptides in microorganisms and eukaryote cells will be made here.

The polypeptide active ingredients of the invention can be purified from the producing host cells by well-known purification methods including ammonium sulfate precipitation, acid extraction, gel electrophoresis, filtration and chromatographic methods (e.g. reverse phase, size exclusion, ion exchange, affinity, phosphocellulose, hydrophobic-interaction, hydroxylapatite, or high performance liquid chromatography). The conditions and technology used to purify a particular peptide or fusion peptide of the invention will depend on factors such as net charge, molecular weight, hydrophobicity, hydrophilicity and will be apparent to those having skill in the art. Moreover, the level of purification will depend on the intended use.

This invention also concerns a pharmaceutical composition comprising an active ingredient selected from the group consisting of:
A) a nucleic acid encoding a polypeptide active ingredient of the invention selected from the group consisting of
   a) a polypeptide comprising an amino acid sequence selected from the group consisting of SEQ ID No 7 and SEQ ID No 8
   b) a polypeptide comprising an amino acid sequence selected from the group consisting of SEQ ID No 7 and SEQ ID No 8.
B) a recombinant vector having inserted therein a nucleic acid as defined in A) above, and
C) a recombinant host cell transformed with (i) a nucleic acid as defined in A) above or with (ii) a recombinant vector as defined in B) above.

The present invention is further illustrated by the examples hereafter.

### EXAMPLES

### Example 1

### Anti-Tumor Activity of Unglycanated Mouse and Human Endocans

### A. Materials and Methods

A.1. Cell culture. The 293, NIH3T3, B16F10, HT-29 cells were routinely cultured in DMEM supplemented with 10% FCS and 2 mM L-glutamine. CHO DG44 were routinely cultured in α-MEM supplemented with 10% FCS, HT-Supp and 2 mM L-glutamine. Hybridoma cells were cultured in RPMI 1640 supplemented with 10% FCS, 5 mM HEPES, HT-Supplement or a serum-free Hybridoma-SFM medium (All culture media purchased by Invitrogen, Cergy Pontoise, France).
A.2. Cloning of mouse endocan. Mouse endocan cDNA cloning. Cloning has been performed by PCR. The first PCR was initiated with primers designed within the 100% homologous sequences between human (X89426) and rat endocan (U80818) sequences (5′-AGAAACTTGCTACCG-3′ [SEQ ID No 11] and 5′-GCCGTAGGGACAGTC-3′ [SEQ ID No 12]). A 125 bp PCR fragment was obtained from BALB/cByJlco (BALB/c) Marathon Ready mouse lung cDNA (Invitrogen, Cergy Pontoise, France). The fragment was cloned in pCR2.1 vector (TA Cloning Kit, Invitrogen, Cergy Pontoise, France) and sequenced with 3730 XL apparatus from Applied Biosystems (Genoscreen, Pasteur Institute of Lille, France). Then 5′ and 3′ rapid amplification of cDNA ends (RACE) was performed from Marathon Ready mouse lung cDNA as recommended by the manufacturer (Invitrogen), cloned in pCR2.1 and sequenced. Finally, the full length mouse endocan sequence was then cloned (GenBank Accession Number AJ249354). Mouse endocan gene cloning. 5′ and 3′ rapid amplification of genomic DNA was performed using cDNA-specific primers. In brief, mouse genomic DNA was extracted from BALB/c splenocytes (Qiagen). The EcoR I-digested genomic DNA was ligated with an adaptor that includes cohesive EcoR I end and specific sequences for PCR-based 5′ and 3′ amplification. The complete mouse esm-1 gene was then cloned (GenBank Accession Number AJ416379).
A.3. Production of recombinants, mutants and chimeric mouse endocan. Accordingly with the human sequence, the serine 138 of the mouse putative O-glycanation site was replaced by an alanine codon by PCR with the Quick-Change site-directed mutagenesis kit, according to the manufacturer's recommendations (Stratagene). Mouse endocan and mouse endocan/S138A cDNAs were amplified with primers including the Hind III and EcoR I restriction sites and inserted into a Hind III-EcoR I-linearized pcDNA3.1 (+) vector (Invitrogen). A chimeric DNA sequence containing the entire reading frame of mouse endocan fused with the Fc domain of human IgG1 was also inserted into pcDNA3.1(+) as previously described above. One microgram of constructs in pcDNA3.1 (+) were transfected into 293, CHO DG44, B16F10 with Fugene (Roche) or Lipofectamine (Invitrogen) for NIH3T3 and HT-29. Stably transfected cells were selected by using G418 (200, 300, 1000 μg/ml for 293, HT-29 and CHO DG44 respectively) and cloned by limit dilution as previously described24.
A.4. mAb generation and characterization. mAb were generated by immunization of lewis rat with purified mouse endocan/Fc from CHO as previously described24. Blast cells from inguinal draining lymph nods were fused with Sp2/0 myeloma cells. Clonal hybridoma cells were screened to recognized mouse endocan/Fc by ELISA. Anti-Fc Abs were coated in carbonate buffer. After blocking, mouse endocan/Fc, endocan/Fc, and CD54/Fc were added. Tested supernatants were incubated, then washed and incubated with HRP-anti rat IgG, washed again and developed with OPD as recommended by the manufacturer. Sixteen hybridoma clones designated GGR were shown to react with mo-endocan/Fc and did not react with CD54/Fc. Different anti-human endocan antibodies were also tested. Among all the anti-endocan mAbs developed in our laboratory, only MEP 14 mAb, previously described as an anti-human endocan C-terminus, recognized also mouse endocan. None other mAbs cross-react between human and mouse endocan. mAbs were purified from cell supernatant as previously described24.
A.S. ELISA. Mouse endocan ELISA were performed as human endocan ELISA with some modifications24. Briefly, MEP 14 (IgG2a/K) was used as a coated mAb (0.5 μg/ml in carbonate buffer) and GGR237 (IgG2a/K, 1 μg/ml) as a sandwich mAb. Mouse endocan standards range from 20 to 0.3 ng/ml. Subsequent incubations with anti-rat IgG2a-conjugated horseradish peroxidase (HRP) (Pharmingen) were followed by revelation with OPD (Sigma) as recommended by manufacturer.
A.6. Mouse endocan characterisation. DEAE-Sepharose: The 293 cell supernatant was passed through a 0.2 cm×1.3 cm DEAE-Sepharose column run originally in 20 mM Tris HCl pH 7.4, containing 0.15 M NaCl (Bio-Rad). Bound endocan was eluted from the DEAE-Sepharose with 20 mM Tris HCl pH 7.4, containing 1 M NaCl, and then concentrated in a 0.5 ml Vivaspin concentrator with a 30 kD molecular weight cut-off (Vivascience). Chondroitinase ABC: The bound DEAE was treated with 1 unit/ml chondroitinase ABC (Sigma) overnight at 37° C. Samples was diluted 1:1 in 2×SDS-PAGE sample buffer and subjected to PAGE and western blot analysis according to standard western blotting procedures using MEP 14 as primary Ab probe, followed by affinity-purified, HRP-conjugated goat anti-mouse Ab (dilution 1:15,000) (Sigma) and an ECL detection kit (Amersham). Reduced conditions were obtained with 0.1 M dithiothreitol. Q-Sepharose To determine the glycanation status of mouse endocan the 293 cell supernatant was passed through a 0.2 cm×1.3 cm Q-Sepharose column run originally in 20 mM Tris HCl pH 7.4, containing 0.05 M NaCl (Bio-Rad). Mouse endocan was eluted from the Q-Sepharose with 20 mM Tris HCl pH 7.4, containing NaCl gradient from 0.1 to 1 M. Each elution fractions were measured by ELISA. The elution fraction were pooled into two major group; The first one corresponded to the elution fractions between 0.1 and 0.4 M NaCl and second one between 0.5 and 1 M NaCl. The first peak was then purified on affinity chromatographic column of MEP 14 for ELISA standard, the second elution group was concentrated in a 0.5 ml Vivaspin concentrator with a 30 kD molecular weight cut-off (Vivascience). Samples were then resuspended in 2×SDS-PAGE sample buffer and subjected to PAGE and western blot analysis. In a second set of experiences, the non glycanated fraction (first elution peak) was evaluated by the elution at 0.4 M NaCl and the glycanated fraction (second elution peak) at 1 M NaCl.
A.7. Mouse models. Animal experiments were performed as described previously 20. Briefly, CB-17 scid/scid homozygous SCID mice (male, 5-6 weeks of age) were injected s.c. into the dorsal interscapular area. Mice received 106 transfected 293 cells or 0.25×10⁶ transfected HT-29 resuspended in 200 μL DMEM without FCS. Twenty-four hours before 293 cells 200 μL of anti asialo-GM1 antibody (Wako Chemicals) were injected intra-peritoneally. Sera were then collected once a week to determine the secretion of mouse endocan by the growing tumour. Mice were also assessed for the presence of palpable tumour once a week. Mice were killed when the tumour volume reached 2 cm³. A systematic macroscopic analysis was realized for each organ and tumour, which were then harvested, fixed in AFA (fixing solution containing Ethanol, Formol and acetic acid—LABONORD Templemars France) and processed for paraffin embedding. Three μm thick paraffin slices were stained with hematoxilin eosin.
A.8. Cell proliferation assays. The cell growth and survival were determined by measuring the BrDU incorporation (Roche) and MTT reduction 29 respectively into HT-29. Cells were seeded at a density of 0.5×10⁴/well in 96-well microplates and cultured during 24 hours in complete medium, including 10% FCS. After 24 hours of starvation in medium without FCS, purified recombinant endocan (human endocan, human endocan/S137A, mouse endocan, mouse endocan/S138A) were added in complete medium. After 24 hours of culture, BrDU incorporation and MTT viability assay were performed recommended by manufacturer. Mitomycine (100 ng/mL) was added for control.

### B. Results

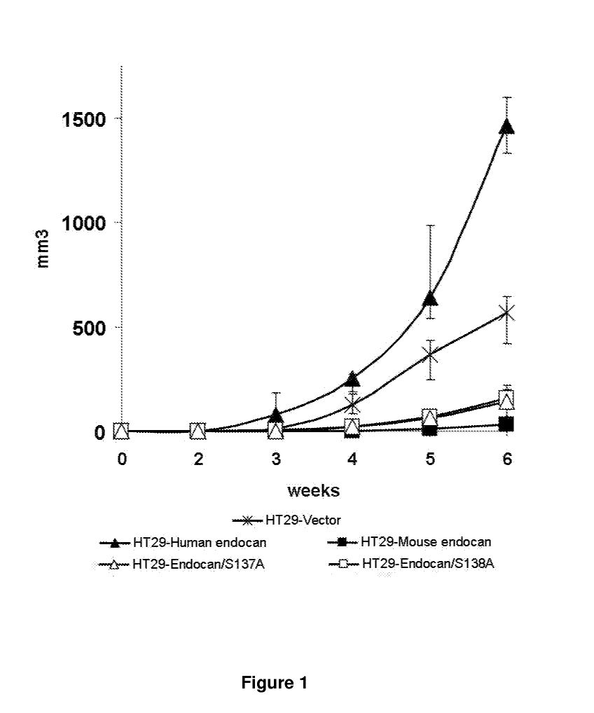

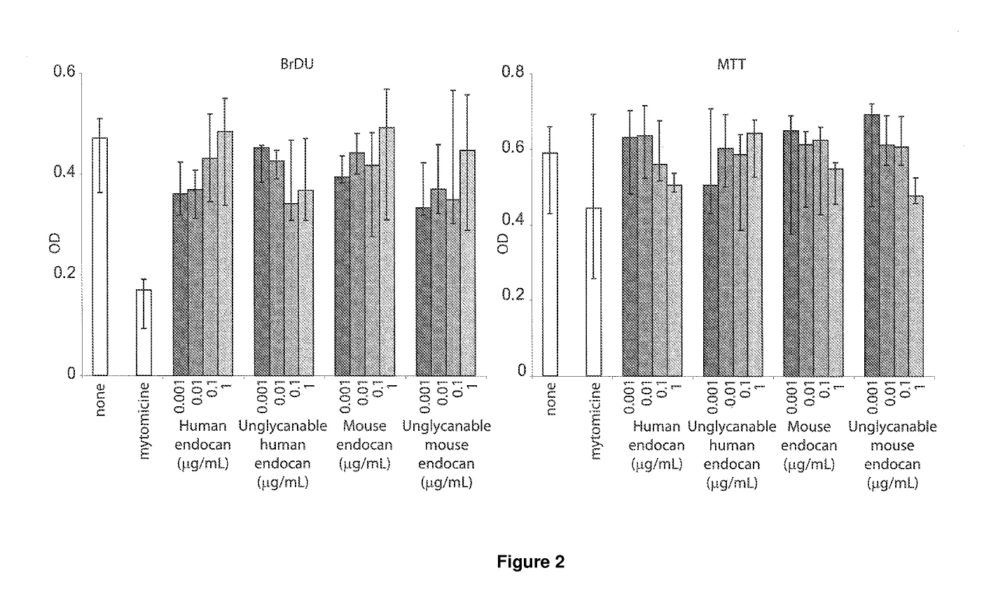

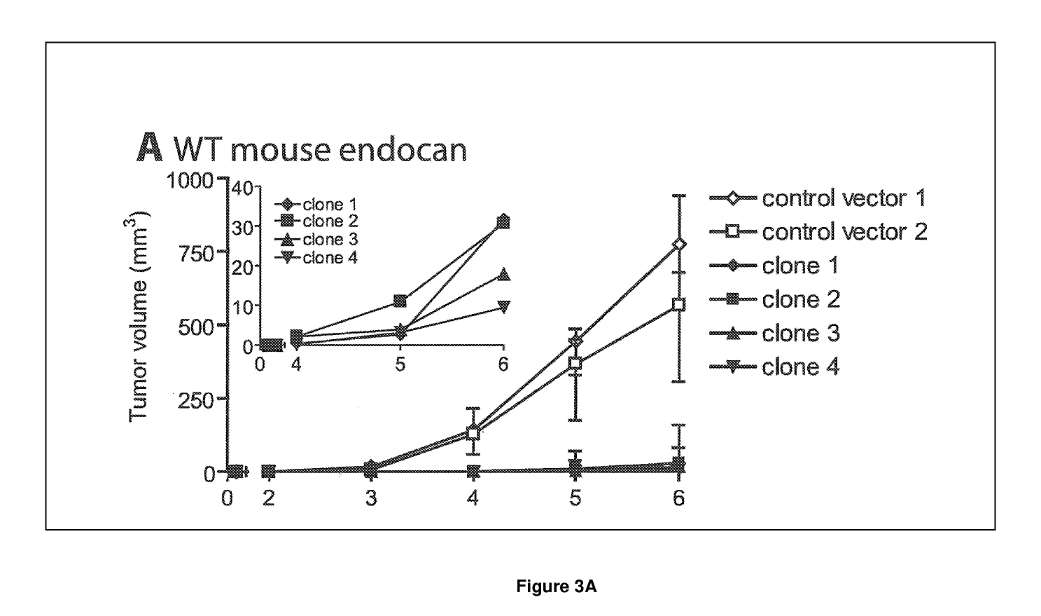

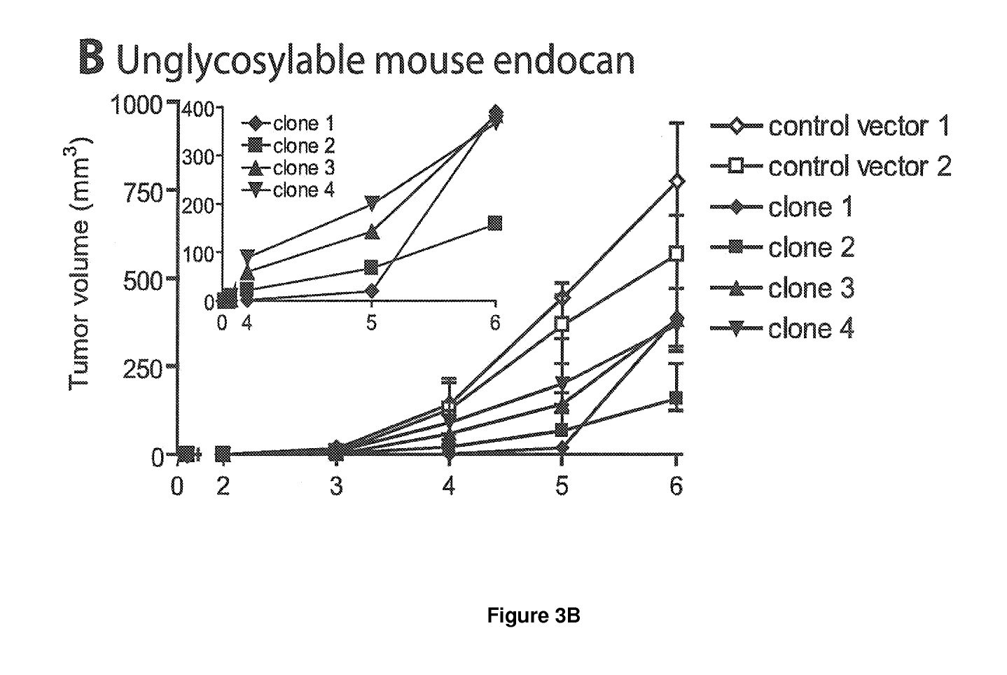

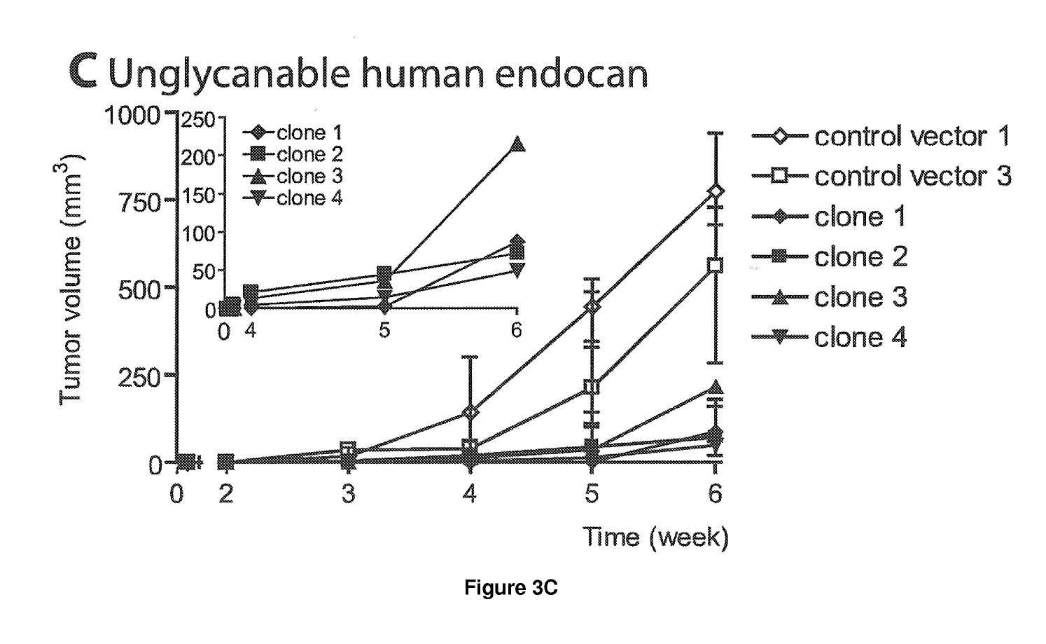

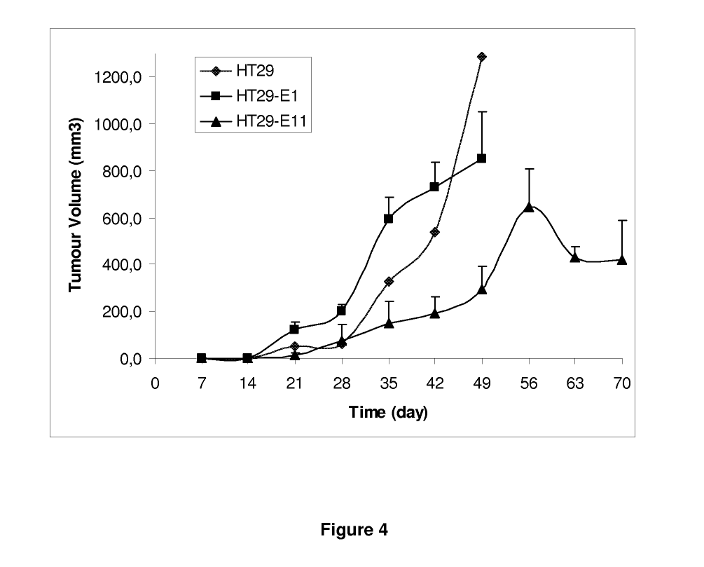

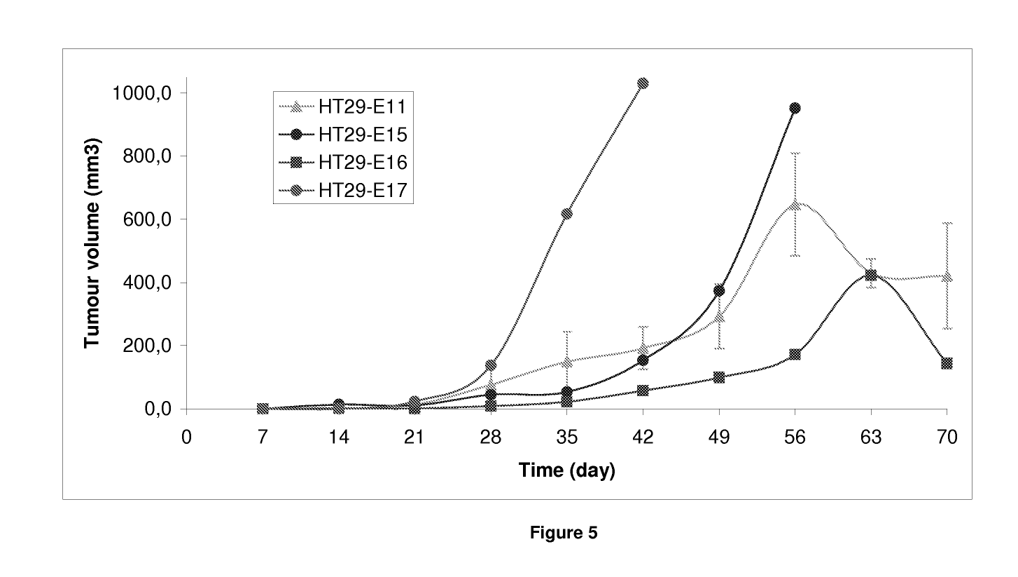

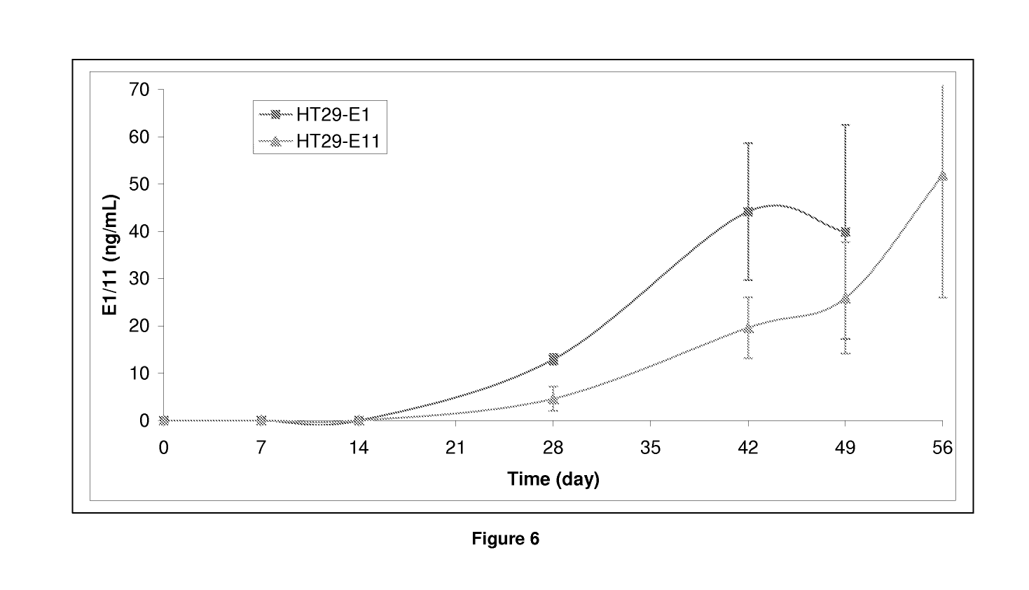

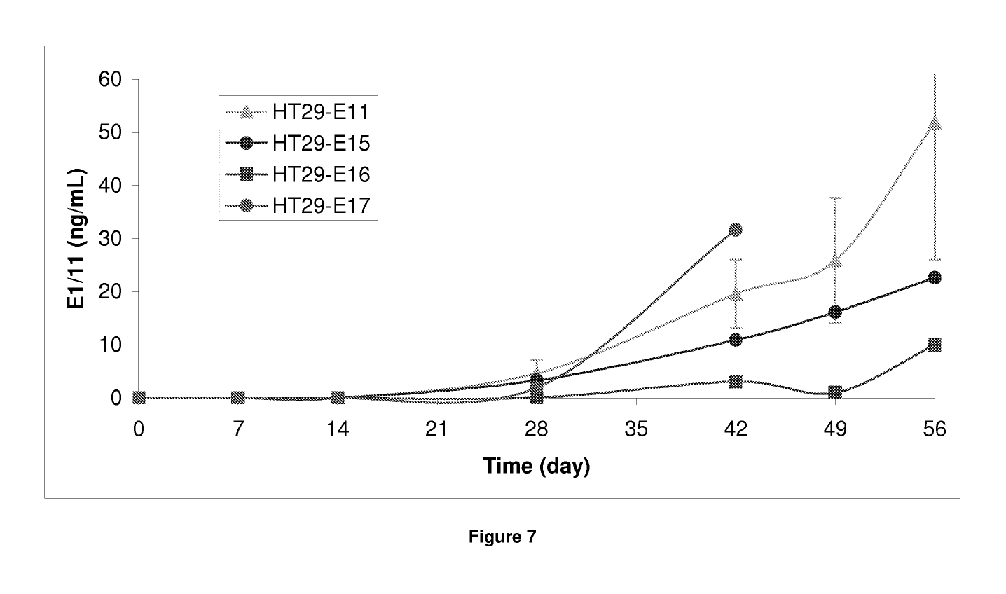

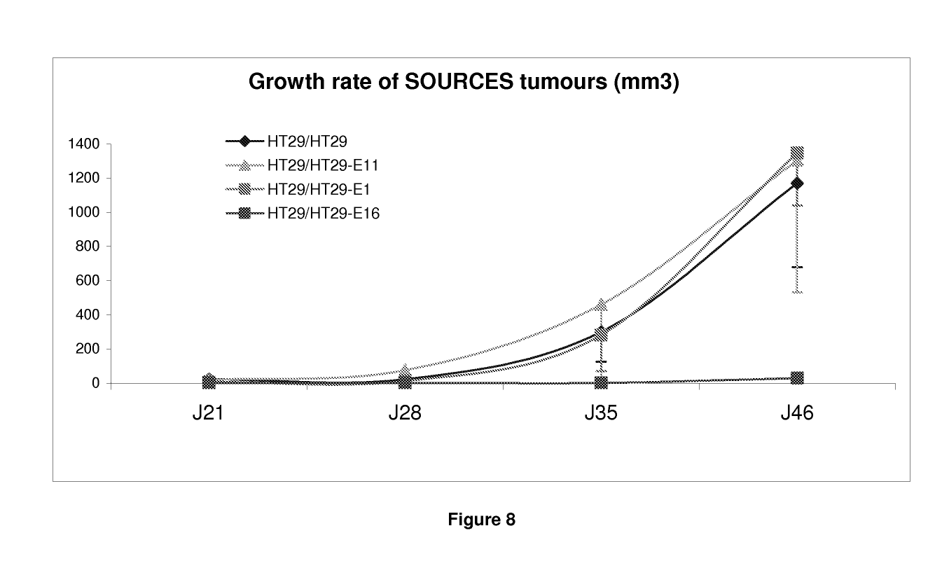

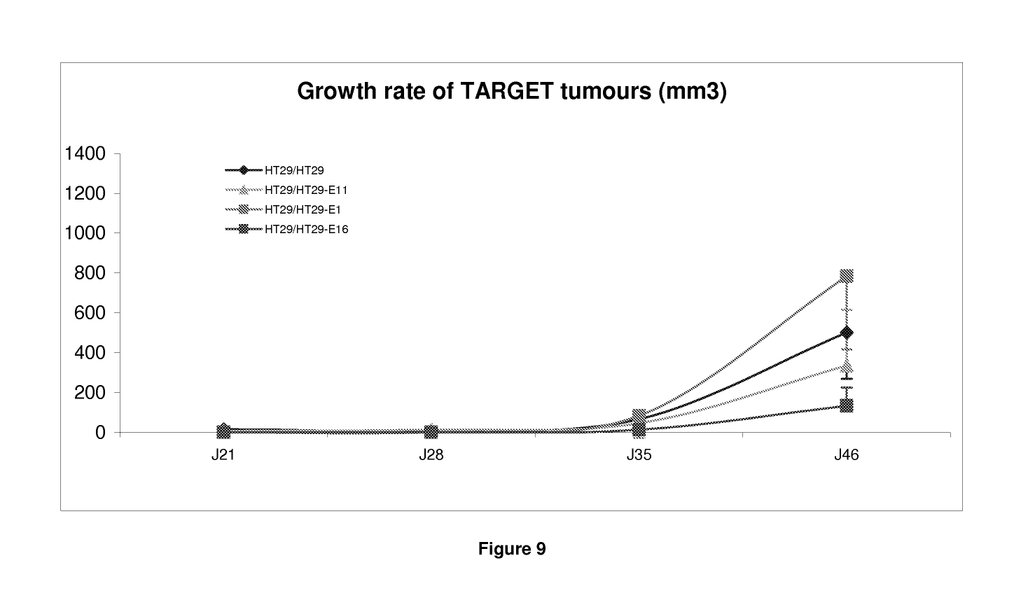

To examine the anti-tumor activity of an unglycanated mouse endocan protein, HT-29 cell clones overexpressing the unglycanable mouse endocan (endocan/S138A) were generated. All tumours exhibited delayed growth rate and smaller tumours than control vector transfected-HT-29 cell tumours (FIG. 1). Next, we wondered if the unglycanable form of human endocan/S137A also exhibited anti-tumour activity. In the same manner, all tumours showed delayed growth rate (FIG. 1). Taken together, these results suggest that the unglycanable form of mouse and human endocan exhibit anti-tumour activity in vivo. This also suggests that the specific amino acids involved in this property are common to both polypeptides. Because of the 75% homology between mouse and human polypeptides, these results emphasize that mouse models of tumour xenograft could be good models to explore human-based anti-tumour activity of unglycanated endocan.

To examine if unglycanated endocan could exert its anti-tumour activity directly, HT-29 cells cultured with various levels of wild type or unglycanable human or mouse endocan, ranging from 1 ng/mL to 1 μg/mL, exhibited no change in BrDU incorporation nor in MTT cytotoxicity assay (FIG. 2). As control the BrDU incorporation is abolished by mitomycin. Thus, there is no convincing data, at least in vitro, that unglycanated endocan induces HT-29 cell cytotoxicity or inhibits HT-29 cell growth, and rather suggests a more indirect effect through modifications of the tumour stroma.

Clinically, a round, subcutaneous tumour was found at the site of cell injection in all cases. When compared with mice having been injected with HT-29 cells, human endocan tumour-expressing mice looked leaner, but the comparison of weight curves did not show any significant difference because the weight of the developing tumour compensated the weight loss of the mice with tumours. At dissection, the tumour did not adhere to the skin or to adjacent organs. Macroscopic and microscopic examination did not show any lymph node or metastatic dissemination. Macroscopic analysis of tumour HT-29 showed a whitish nonadherent nodule, with necrotic areas. Percentage of necrotic areas did not differ between different tumour types. A more abundant stroma and a significant leukocytic infiltrate at the tumour periphery were present in tumours expressing mouse endocan or mouse unglycanable endocan or human unglycanable endocan compared to parental HT-29 tumours or human endocan.

In summary the results that have been obtained show that the endogenous stromal mediator endocan affects tumour growth. It has thus been shown that an unglycanated form of a human endocan or of a mouse endocan reveals a valuable active ingredient for anti-tumour immunotherapy.

### Example 2

### Anti-Tumor Activity of Various Unglycanated Human Endocans

### A. Materials and Methods

### A.1. Materials.

The pcDNA3 vector that contains the wild type endocan or the non glycanated endocan cDNA, has been mutated in order to replace either F115 or F116 or both by Alanine residues using the Quick Site Directed Mutagenesis Kit (Stratagene), and their sequences verified on ABI prism apparatus (Genoscreen, Lille, France).
The different constructs were called:
E1 for endocan (wild type),
E11 for S137A endocan=non glycanated endocan,
E12 for F115A endocan
E13 for F116A endocan
E14 for F115A and F116A endocan
E15 for F115A non glycanated endocan,
E16 for F116A non glycanated endocan,
E17 for F115A and F116A non glycanated endocan.
The cDNAs were transfected in HT-29 using lipofectamine reagent and then selected by addition of 300 μg/mL G418. The cells were then subcultured by limited dilution in the presence of G418 and clones were selected on the detection of endocan in their supernatants using proprietary specific ELISA. The cells were characterized by their levels of endocan production. The mycoplasma-free cell clones were stored in the master cell bank of U774 (Inserm U774, Pasteur Institute of Lille, Lille, France).

### A.2. Mouse Models.

Briefly, CB-17 scid/scid homozygous SCID mice (male, 5-6 weeks of age) were injected s.c. into the dorsal interscapular area. Mice received 2×10⁵ transfected HT-29 resuspended in 200 μL DMEM. Sera were then collected once a week to determine the secretion of mouse endocan by the growing tumour. Mice were also assessed for the presence of palpable tumour once a week. Mice were killed when the tumour volume reached 2 cm³. A systematic macroscopic analysis was realized for each organ and tumour, which were then harvested, fixed in AFA (fixing solution containing Ethanol, Formol and acetic acid—LABONORD Templemars France) and processed for paraffin embedding. Three μm thick paraffin slices were stained with hematoxilin eosin (A Janin, Inserm U728, Paris, France).

### B. Results

### B.1. Non-Glycanated Human Endocan Mutated on Serine 137 Inhibits the Growth of Tumor Xenografts.

Subcutaneous injection of either HT29 cells overexpressing E1 (here only called BL10) or E11 in SCID mice resulted in formation of tumours clinically palpable between the 3rd and the 4th week. However, there were important differences in the growth rate of the tumours: As shown in FIG. 3, the HT29 cells expressing E1 grew more rapidly than the parental cells. On the other hand the HT29 cells overexpressing E11 (the non glycanated endocan) grew less quickly than HT29 overexpressing E1 and also less quickly than the parental HT29 cells.
Pathological analysis of the tumours revealed that both HT29 and HT29-E1 tumours contains almost tumor cells and a very weak stroma only containing blood vessels. By contrast, the HT29-E11 tumours contained tumour cells and a significant stromal inflammatory reaction comprising blood vessels, perivascular leucocytes and fibrosis. Immunohistochemistry showed that human endocan is exclusively produced by tumour cells and that mouse endocan is exclusively detected in tumor vessels.

### B.2. Showing of the Absence of a HT29 Clonal Bias

Three separate HT29 cell clones overexpressing E1 or E11 were subcutaneously injected into SCID mice (2×10⁵ cells per mouse, 4 mice per clone) (Classeur des clones). Mice were examined each. Mice were sacrified at week 7 and tumours analysed microscopically.
The results shown in FIG. 4 indicated that the growth rates were similar for each clone overexpressing the same molecule. Specifically, the slow growth rate of HT29-E11 was regularly observed with each clone, indicating that the growth rate was not due to a clonal bias but rather due to the recombinant molecule produced by the cells. Mean+/−SD of mean of the 3 clones (12 mice). Histologically, only HT29-E11 tumours exhibit stromal inflammatory reaction (as histological figure just above).

### B.3. Critical Role of Phenylalanine Residues in Positions 115 and 116 on the Anti-Tumor Activity of Human Unglycanated Endocan.

### B.3.1. Differential Role of F115 and F116 in Anti-Tumor Activity

Three HT29 cell clones overexpressing E11, E15, E16 or E17 were subcutaneously injected in SCID mice (4 mice per clone).
The results are shown in FIG. 5. The growth rate of HT29-E17 is similar to that of parental HT29 cells: both F115 and F116 are required for anti-tumour activity of E11. The growth rate of HT29-E15 is similar to that of HT29-E11:F115 does not appear critical for anti-tumour activity, which could be compensated by F116. The growth rate of HT29-E16 is slower than that of HT29-E11: F115 plays a critical role in relationship with F116.

### B.3.2. Blood Levels of Human Endocan in Tumor-Bearing Mice.

The results are shown in FIG. 6 and FIG. 7.
Blood E1, E11, E15, E16, and E17 are detected in levels ranging from 5 to 50 ng/mL. These levels increase with time in part related to the increasing size of the tumours

### B.3.3. Induction of a Marked Stromal Inflammation by Unglycanated Endocans

It has been found that HT29-E16 tumours exhibit an intense stromal remodelling which contains an pan-leukocytic infiltrate, fibroblats, fibrosis and tumour vessels (HE×100). Endocan binds to LFA-1 and inhibit ICAM-1-LFA-1 interaction. It is thus believed that E11 and its more efficient derivative E16 act as competitive antagonists for the endocan's receptor LFA-1. The highest efficiency of E16 might be explained by its greater affinity for LFA-1.

### B.4. Systemic Anti-Tumor Activity of Unglycanated Endocans.

The above results showed that local overexpression of E16, within the tumour, slowed considerably the growth rate of the HT29 tumour xenografts by comparison to that of parental HT29 tumours. In order to study the effect of systemic administration of E16 on HT29 tumours, we have developed the double tumour model which consists in simultaneous injection of SOURCE HT29-E16 cells into right scapular region and TARGET parental HT29 cells in the controlateral back.
1/The double tumour model. The principle was to examine if E16, given through a systemic pathway, is able to reduce the growth rate of HT29 tumour xenografts. Because blood endocan levels were significant during the follow up of the transfected HT29 tumour xenografts, we tested if this blood endocan could act on parental HT29 tumours which did not produce endocan. The results showed that SOURCE tumours grew as expected (See FIG. 8). Interrestingly, the growth rate of TARGET tumours slowed down a little bit in the presence of blood E11, and slowed down more importantly in the presence of blood E16 (See FIG. 9).
The SOURCE and TARGET tumours were microscopically examined (Data not shown). As expected, a stromal inflammatory reaction was observed only in HT29-E11 and HT29-E16 tumours. Surprisingly, such an inflammatory reaction was observed in parental HT29 tumours in the presence of blood E16.

TABLE 1
Sequence References

| SEQ ID N^{°} | Type | Designation |
|---|---|---|
| 1 | Peptide | Secreted human endocan |
| 2 | Peptide | Secreted mouse endocan |
| 3 | Peptide | Human endocan |
| 4 | peptide | Mouse endocan |
| 5 | Peptide | Unglycanated human S137A endocan |
| 6 | Peptide | Unglycanated mouse S137A endocan |
| 7 | Peptide | Unglycanated human F116A, S137A |
| | | endocan |
| 8 | Peptide | Unglycanated mouse F116A S138A |
| | | endocan |
| 9 | Nucleic acid | Human endocan |
| 10 | Nucleic acid | Mouse endocan |
| 11 | Nucleic acid | Primer |
| 12 | Nucleic acid | primer |

1-11. (canceled)

12. A method of treating a cancer, comprising administering to a subject in need thereof a non-glycanated form of a polypeptide comprising an amino acid sequence having at least 90% amino acid identity with an amino acid sequence selected from the group consisting of SEQ ID No 1 and SEQ ID No 2.

13. The method according to claim 12, wherein said polypeptide comprises an amino acid sequence selected from the group consisting of:
a) an amino acid sequence having at least 90% amino acid identity with the amino acid sequence of SEQ ID No 1 and wherein the serine amino acid residue in position 137 of SEQ ID No 1 is replaced by a distinct amino acid residue; and
b) an amino acid sequence having at least 90% amino acid identity with the amino acid sequence of SEQ ID No 2 and wherein the serine residue in position 138 of SEQ ID No 2 is replaced by a distinct amino acid residue,

14. The method according to claim 12, wherein said polypeptide comprises an amino acid sequence selected from the group consisting of:
a) an amino acid sequence having at least 90% amino acid identity with the amino acid sequence of SEQ ID No 1 and wherein (i) the serine amino acid residue in position 137 of SEQ ID No 1 is replaced by a distinct amino acid residue and (ii) the phenylalanine residue in position 116 of SEQ ID No 1 is replaced by a distinct amino acid residue; and
b) an amino acid sequence having at least 90% amino acid identity with the amino acid sequence of SEQ ID No 2 and wherein the serine residue in position 138 of SEQ ID No 2 is replaced by a distinct amino acid residue and (ii) the phenylalanine residue in position 116 of SEQ ID No 2 is replaced by a distinct amino acid residue,

15. The method according to claim 12, wherein said polypeptide comprises an amino acid sequence selected from the group consisting of SEQ ID No 5 and SEQ ID No 6.

16. The method according to claim 12, wherein said polypeptide comprises an amino acid sequence selected from the group consisting of SEQ ID No 7 and SEQ ID No 8.

17. A non-glycanated comprising an amino acid sequence selected from the group consisting of SEQ ID No 7 and SEQ ID No 8.

18. A pharmaceutical composition comprising a polypeptide comprising an amino acid sequence selected from the group consisting of SEQ ID No 5, 6, 7 and 8.

19. A nucleic acid encoding a polypeptide according to claim 17.

20. A recombinant vector having inserted therein a nucleic acid according to claim 19.

21. A recombinant host cell transformed with a nucleic acid according to claim 19 and expressing a polypeptide comprising an amino acid sequence selected from the group consisting of SEQ ID No 5, 6, 7 and 8.

22. A recombinant host cell transformed with a recombinant vector according to claim 20 and expressing a polypeptide comprising an amino acid sequence selected from the group consisting of SEQ ID No 5, 6, 7 and 8.

23. A pharmaceutical composition comprising an active ingredient selected from the group consisting of:
a) a nucleic acid according to claim 19,
b) a recombinant vector having inserted therein a nucleic acid according to claim 19,
c) a recombinant host cell transformed with a recombinant vector
having inserted therein a nucleic acid according to claim 19, and expressing a polypeptide comprising an amino acid sequence selected from the group consisting of SEQ ID No 5, 6, 7 and 8.

24. A pharmaceutical composition comprising an active ingredient selected from the group consisting of:
a) a nucleic acid according to claim 19,
b) a recombinant vector having inserted therein a nucleic acid according to claim 19,
c) a recombinant host cell transformed with a nucleic acid according to claim 19 and expressing a polypeptide comprising an amino acid sequence selected from the group consisting of SEQ ID No 5, 6, 7 and 8.
